(19) United States
(12) Patent Application Publication
(10) Pub. No.: US 2012/0129243 A1
(43) Pub. Date: May 24, 2012

(54) Algae Producing Trough System

(75) Inventors: George Benjamin Cloud, Chandler, AZ (US); Souren Naradikian, Phoenix, AZ (US); Steve Irwin, Avondale, AZ (US)

(73) Assignee: PHYCO BIOSCIENCES, INC., Chandler, AZ (US)

(21) Appl. No.: 13/111,862

(22) Filed: May 19, 2011

Related U.S. Application Data
(62) Division of application No. 12/436,583, filed on May 6, 2009.
Application No. 12/436,583 is a continuation-in-part of application No. 12/156,506, filed on Jun. 2, 2008, now abandoned.
(60) Provisional application No. 60/932,674, filed on May 31, 2007.
(60) Provisional application No. 61/126,701, filed on May 6, 2008.

(51) Int. Cl. C12N 1/12

(52) U.S. Cl. 435/257.1

(57) ABSTRACT

A trough lining assembly is placed in a series of troughs at a biomass processing facility. The trough lining assembly includes a waterproof liner that lies against the sides of a trough, an aerator, and a retention mechanism that retains the aerator at the bottom of the trough. The aerator provides continuous aeration of biomass present in the trough by releasing aerating gas into the biomass along the length of the trough. The continuous aeration also churns the biomass, exposing more of it to the aerating gas and to sunlight. The trough lining assembly improves the efficiency of algae production by stimulating photosynthesis and consumption of carbon dioxide. The trough lining assembly has low production, transportation, installation, and maintenance costs.

### CROSS-REFERENCE TO RELATED APPLICATIONS

This application is a divisional application of co-pending U.S. patent application Ser. No. 12/436,583, filed May 6, 2009, which is a nonprovisional application of U.S. Pat. App. Ser. No. 61/126,701, filed May 6, 2008, and which also claims the benefit and is a continuation-in-part of U.S. patent application Ser. No. 12/156,506, filed Jun. 2, 2008, which claims the benefit of provisional application No. 60/932,674, filed May 31, 2007.

### FIELD OF INVENTION

This application relates to mechanical aeration of a biomass. This application relates particularly to a method and apparatus for simultaneously aerating and circulating a biomass to stimulate chemical changes therein.

### BACKGROUND

Microscopic algae are unicellular organisms which produce oxygen by photosynthesis. Microscopic algae, referred to herein as algae, include flagellates, diatoms and blue-green algae; over 100,000 species are known. Algae are used for a wide variety of purposes, including the production of vitamins, pharmaceuticals, and natural dyes; as a source of fatty acids, proteins and other biochemicals in health food products; as an animal feed supplement with nutritional value equivalent to that of soybean meal; for biological control of agricultural pests; as soil conditioners and biofertilizers in agriculture; the production of oxygen and removal of nitrogen, phosphorus and toxic substances in sewage treatment; in biodegradation of plastics; as a renewable biomass source for the production of a diesel fuel substitute (biodiesel) and other biofuels such as ethanol, methane gas and hydrogen; to scrub CO₂, NOₓ, VOₓ from gases released during the production of fossil fuel; and as animal feed. With so many uses, it would be desirable to mass produce algae in a low-cost, high-yield manner.

Algae use a photosynthetic process similar to that of higher-developed plants, with certain advantages not found in traditional crops such as rapeseed, wheat or corn. Algae have a high growth rate; it is possible to complete an entire harvest in hours. Further, algae are tolerant to varying environmental conditions, for example, growing in saline waters that are unsuitable for agriculture. Because of this tolerance, algae are responsible for about one-third of the net photosynthetic activity worldwide. Cultivation of algae in a liquid environment instead of dirt allows them better access to resources: water, CO₂, and minerals. It is for this reason that the algae are capable, according to scientists at the National Renewable Energy Laboratory, “of synthesizing 30 times more oil per hectare than the terrestrial plants used for the fabrication of biofuels.” (John Sheehan, et al.) The measurement per hectare is used because the important factor in the algae's cultivation is not the volume of the basin where they are grown, but the surface exposed to the sun. Productivity is measured in terms of biomass per day and by surface unit. Thus, comparisons with terrestrial plants are possible. Professor Michael Briggs at the University of New Hampshire estimates that the cultivation of these algae over a surface of 38,500 km², and situated in a zone of high sun-exposure such as the Sonoran Desert, would make it possible to replace the totality of petroleum consumed in the United States. Interest in the biotechnology is therefore immense. Arid zones are ideal for the cultivation of algae because sun exposure is optimal while human activity is virtually absent. These algae can be nourished on recycled sources such animal manures. Presently, research is being done on algae that are rich in oils and whose yield per hectare is considerably higher than other oilseed crops such as corn and rapeseed. NREL and the Department of Energy are working to produce a commercial-grade fuel from triglyceride-rich micro-algae. NREL has selected 300 species of algae, both fresh water and salt water algae, including diatoms and green algae, for further development.

Yield can be limited by the limited wavelength range of light energy capable of driving photosynthesis, between about 400-700 nm, which is only about half of the total solar energy. Other factors, such as respiration requirements during dark periods, efficiency of absorbing sunlight, and other growth conditions can affect photosynthetic efficiencies in algal bioreactors. The net result is an overall photosynthetic efficiency that has been too low for economical large scale production. The need exists for a large scale production system that provides the user a cost-effective means of installation, operation and maintenance relative to production yields. It is desirable that such a system also increase photosynthesis to maximize production yield.

In order to produce optimal yields, algae need to have CO₂ in large quantities in the basins or bioreactors where they grow. However, known systems employ inefficient processes of aerating the algae with CO₂. Typical open-pond or basin systems have a single injection point for the CO₂, which is expect to diffuse throughout the biomass. The pond or basin is very large, however, and even if the CO₂ successfully diffuses throughout the biomass, it takes a very long time to do so. Another solution is the raceway system, wherein a paddle wheel pushes the biomass around a track. Again, a single point of injection, typically near the paddle wheel, “loads” the biomass with CO₂. The CO₂ is consumed long before the algae again reach the injection point, resulting in a period of time when the algae is not growing as fast as it could be. Closed bioreactor systems employ similar CO₂ loading techniques, where one or multiple injection points load CO₂ that is completely consumed by the biomass before it reaches the next injection point. It would be advantageous to maximize contact between the CO₂ gas and the developing algae by providing continuous aerating of the algae biomass.

One proposal for a large-scale bioreactor system uses a series of rigid pipes elevated over an earthen bed. This system suffers some disadvantages, however, because the rigid pipes are expensive to transport and difficult to install and maintain. Another approach uses polyethylene tubes coupled to a rigid roller structure. The flexible bioreactor tubes are made of two layers of 10 mil thick polyethylene, and lay between the two sets of guard rails. Rollers traverse the tubes to peristaltically move the algae through the bags. In one attempt to avoid an outdoor facility, the Japanese government has launched a research program to investigate the development of reactors which would use fiber optic lighting which would reduce the surface area necessary for algae production and ensure better protection against variety contamination. Unfortunately, all these approaches suffer the same significant disadvantage: they require a framework or other rigid structure be built to operate the system. It would be advantageous to avoid having to build a structure or framework, or at least minimize the amount of building required, in order to minimize capital cost, and reduce the difficulty in erecting and maintaining an algae system.

Another disadvantage of rigid systems is that the accumulation of gases resulting from algae production may restrict the flow of the biomass through the system. Algae consume CO₂ and produce O₂ and water vapor. A rigid system cannot expand in response to the increasing volume of gas within the system; as the pressure increases, the gases restrict the flow through the system and affect harvesting. Further, the system may eventually exceed a maximum pressure and rupture, resulting in repair and downtime costs. Simply installing pressure release valves would negate the potential benefits of collecting the gases, such as measuring the efficiency of CO₂ absorption and harvesting pure oxygen for burning or other uses. A system that accommodates the expanding gas volume and allows for maximum collection of the gases is desired.

Therefore, it is an object of this invention to provide a large-scale algae production system. It is another object to provide an algae production system that has a lower capital cost than elevated rigid piping and other existing systems. It is another object to simplify installation and maintenance of an algae system. It is another object to increase efficiency of an algae production system by exposing more algae to light and CO₂. It is another object to facilitate collection of gases in the system without restricting biomass flow.

### SUMMARY OF THE INVENTION

The invention is a trough system for aerating a biomass, such as one containing algae. The system comprises a trough lining assembly that conforms to the shape of a trough dug into the ground. A reinforced polymer liner lies on the trough walls and may be attached to the trough or held in place by the weight of the biomass. An aerator releases aerating gas into the biomass, churning and aerating the biomass. The aerator may be retained at the bottom of the trough by adhesion to the liner or attachment to the liner by heat seal during the manufacturing process, but preferably a retaining strip is attached to the liner and the aerator is retained in the envelope between the liner and the retaining strip. A self-luminescent material may be applied to the liner or the envelope to provide growth-inducing light continuously. A solar cover may be laid above the trough for control of temperature, moisture and light exposure. The solar cover may be expandable to accommodate the accumulation of gases inside the lined trough.

In a multiple-trough system, the trough lining assemblies are connected to a common inlet and outlet line, a circulation pump, control valves, and a gas injector. A biomass is deposited into the trough assemblies. Aerating gas is injected under pressure into the trough assemblies, so that the aerating gas is released through the aerator, preferably in the form of microbubbles. As the biomass is circulated through the trough lining assemblies, the aerating gas continuously aerates the biomass while causing a motive force that churns the biomass. Where the biomass contains algae, the continuous churning increases the amount of algae that is exposed to sunlight and the aerating gas. By exposure to sunlight, the algae photosynthesize, consuming CO₂, producing O₂, and reproducing. Once the algae biomass is concentrated enough to harvest, the biomass is gradually diverted into a harvesting system to extract the algae from the biomass. The O₂ produced during photosynthesis may be collected through gas collection valves. Where the system is connected to a power plant or other production facility, the collected gas can be analyzed to determine reduction of CO₂ emissions and reintroduced into the facility to increase efficiency of combustion machinery. The algae may be dried onsite using an integrated dryer.

### BRIEF DESCRIPTION OF THE DRAWINGS

FIG. 1a is a cross-section schematic of the preferred embodiment of a trough lining assembly in a trough containing biomass, also showing a tractor in a service position.

FIG. 1b is a cross-section schematic of a portion of the preferred embodiment of a trough lining assembly in a trough, as in FIG. 1a, with a solar cover laid on the surface of the biomass.

FIG. 2a is a cross-section schematic of a portion of an alternate embodiment of a trough lining assembly in a trough, also showing a tractor in a service position and a solar cover covering the trough and held in place by berms.

FIG. 2b is a cross-section schematic of a portion of the alternate embodiment of a trough lining assembly of FIG. 2a, after accumulation of gases.

FIG. 3a is a top-view schematic of a portion of the preferred embodiment of a trough lining assembly in a trough, not containing biomass, showing a pattern of air openings in the envelope.

FIG. 3b is a top view of an envelope with a pattern of slits as air openings.

FIG. 3c is a top view of an envelope with a pattern of rounded rectangles as air openings.

FIG. 3d is a top view of an envelope with a pattern of squares as air openings.

FIG. 4 is a cross-section schematic of a portion of the preferred embodiment of a trough lining assembly in a trough containing biomass, showing the motive force imparted by rising aerating gas.

FIG. 5 is a side view of the preferred embodiment of a trough lining assembly rolled onto a deploying spool.

FIG. 6 is a top view of the preferred embodiment of a field.

FIG. 7a is a top view of an alternate embodiment of a field.

FIG. 7b is a top view of three troughs in the field of FIG. 7a.

FIG. 8 is a top view of an on-site algae dryer.

### DETAILED DESCRIPTION OF THE INVENTION

This invention uses a trough liner assembly that lies within a trough. The trough liner assembly aerates and circulates a biomass deposited therein. The biomass may be any biomass that receives a chemical benefit from aeration and circulation. For example, the invention serves as an aerobic digester for the treatment of wastewater, and stimulates growth of algae, shrimp, fish, and other water-based biological products. Algae and other plants may benefit the most from the invention, as the invention promotes exposure of the biomass to light and aerating gas. Multiple troughs are arranged side-by-side to form a bed. One or more beds form a facility of sufficient capacity to meet the local needs when the sunlight is at its most limited, and excess capacity with greater sunlight.

Beds

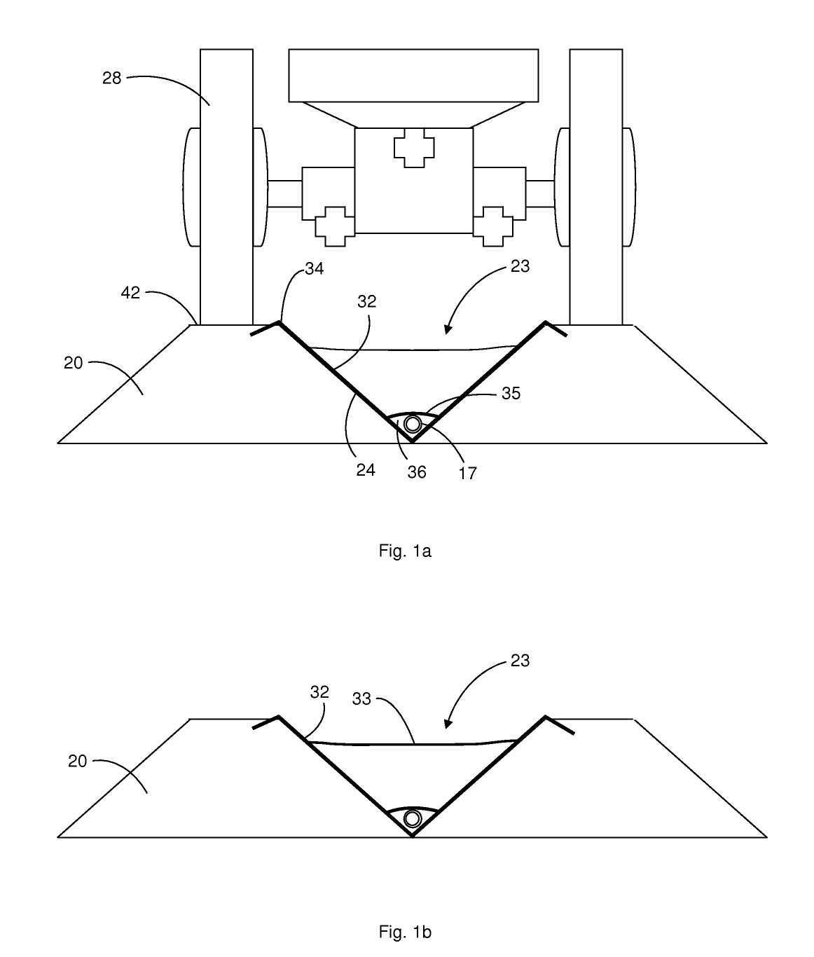

A trough 23 is formed by either digging into the ground or by shaping loose dirt into berms 20 that form the trough sides 24. See FIG. 1a. The berms 20 are preferably 110 inches apart, measured from center to center of each berm 20. A trough 23 is preferably over 350 feet long, most preferably 1250 feet long, and v-shaped, having two substantially planar sides 24 which converge to a point at the bottom, and an open top. In the preferred embodiment, the depth of the trough 23 is at least 24 inches, measured as the vertical distance from the bottom of the trough 23 to the horizontal line stretched between the tops of the berms 20 that form the trough sides 24. The width of the trough 23 is the distance between the trough sides 24 measured at the top of the berms 20 that form the trough sides 24. Preferably, the width is 80 inches. In a trough 23 having the preferred dimensions, the biomass in the trough will preferably have a depth of 18 inches and a width at fill level of 60 inches. The depth and width of the trough 23 may vary depending on the amount of expected rainfall in the region, the composition of the biomass, and the desired effect of the aeration. For example, the preferred dimensions are known to stimulate growth in Chlorella and Nanochloropsis varities of algae, but the trough 23 should be substantially wider to grow fish or shrimp, and may be narrower and deeper to treat wastewater. The trough 23 may alternatively be any other shape that facilitates aeration of a biomass, including but not limited to u-shaped, concave, rectangular, or asymmetrical.

A berm 20 separates two troughs 23 and has a flat top forming a tractor path 42 wide enough for a tractor 28 to drive over. The tractor 28 requires two tractor paths 42, one on each side of the trough 23, so that it straddles the trough 23 to service it. Preferably, the tractor path 42 is 30 inches wide. A bed is created by forming troughs 23 side-by-side with a tractor path 42 between each trough 23, covering a field 1. Each trough 23 contains a trough lining assembly which transports the biomass through the system.

Trough Lining Assembly

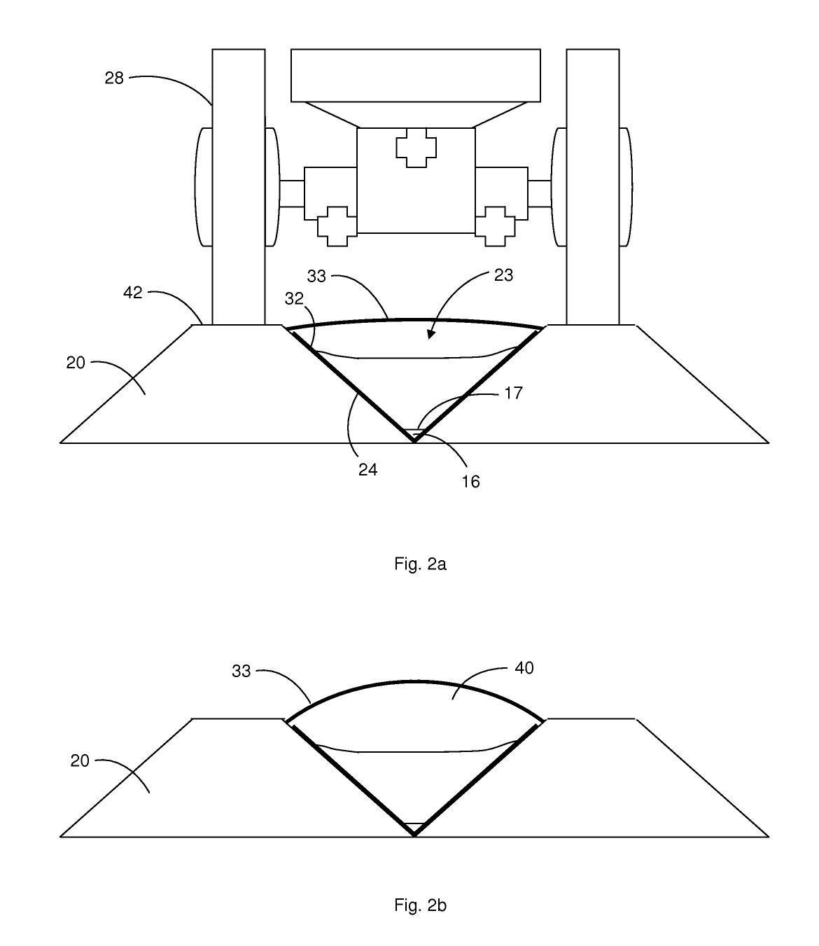

Referring to FIGS. 1a-2b, a trough lining assembly has a single sheet of a thin-walled, waterproof liner 32 along the sides of the trough 23. Preferably the liner 32 is made of reinforced polyethylene that is at least 10 mil thick, but more preferably is at least 12.5 mil thick. The liner 32 may be held in place by the weight of the biomass introduced into the trough lining assembly, or the liner 32 may be retained against the trough sides 24 by other means. In the preferred embodiment, the liner 32 extends up the trough sides and the ends of the liner 32 are covered by the berms 20 to a distance sufficient for the weight of the berm 20 to hold the liner 32 in place. The portion of the liner 32 that is above the level of the biomass, referred to herein as the “hip” 34, may suffer quicker degradation than the rest of the liner 32 due to its exposure to the sun. The hip 34 may be treated with a protective material, such as a layer of reflective paint or self-luminescent material that is introduced into the liner 32 during the manufacturing process or applied to the liner 32 once it is laid in place in the trough 23. Preferably, the self-luminescent material comprises Litroenergy™ self-luminescent micro particles, manufactured by MPK Co. Litroenergy™ particles are non-toxic and crush-resistant up to 5000 lbs., and provide continuous light for a half-life of 12 years without exposure to sunlight. In addition to the hip 34, some or all of the remaining surface of the liner 32 may contain or be covered by the self-luminescent material, in order to stimulate algae growth when sunlight is diminished or absent. For example, horizontal stripes of Litroenergy™-infused paint may be applied to the liner 32 so that the stripes sit below the level of the biomass once the trough lining assembly is in place in the trough 23.

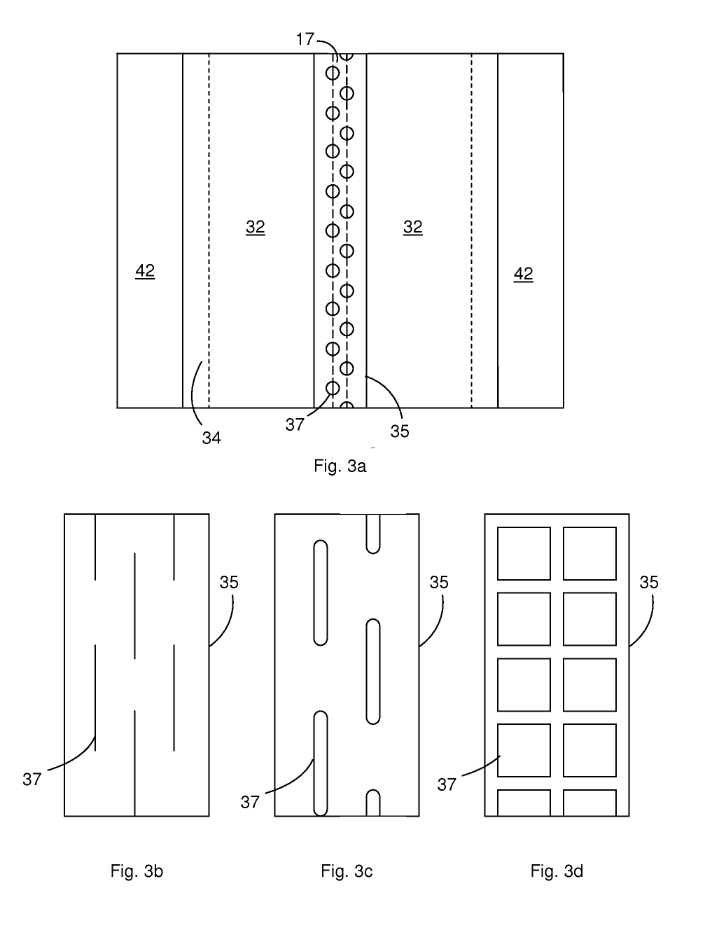

The trough lining assembly also has an aerator 17 that emits aerating gas injected into the assembly. The aerator 17 cooperates with the liner 32 to aerate and churn the biomass in the trough, as described below. The aerator 17 may be perforated or porous, so that the aerating gas passes through it into the biomass. Preferably, the aerator 17 is a porous material made of spun polyethylene fiber, such as Tyvek®. The pores in such a material are so small that the aerating gas will not pass through it until a certain air pressure is reached, at which point the aerating gas is released in the form of microbubbles. Generally, no more than 2 psi of air pressure is required to produce microbubbles. Where algae or other plants are present in the biomass, the aerating substrate 17 preferably releases the aerating gas at a rate that allows substantially all of the gas to be absorbed within the biomass before it reaches the top of the trough 23. The rate of release through the aerator 17 can be limited by using different porosities of Tyvek® or other materials, or by coating the aerator 17 with varying thicknesses of porous or non-porous material. In the preferred embodiment, shown in FIGS. 1a, 1b, and 3a, the aerator 17 is a pressurizable Tyvek® tube that lies flat when it is not pressurized.

The aerator 17 may be positioned at or near the bottom of the trough 23 so that the released aerating gas rises through and is diffused within the biomass. Because the aerator 17 may be buoyant with respect to the biomass, particularly when it is pressurized with aerating gas, a retention mechanism may be used to retain the aerator 17 at or near the bottom of the trough 23. The retention mechanism may be any mechanism that retains the aerator 17 at or near the bottom of the trough 23, without damaging the liner 32, aerator 17, or biomass. For example, the retention mechanism may be a series of weights attached to the aerator 17, a series of fibrous loops surrounding the aerator 17 and attached to the liner 32, or a retaining strip positioned above the aerator. In the preferred embodiment, shown in FIGS. 1a, 1b, and 3a, a retaining strip 35 forms an envelope 36 for retaining the aerator 17 between the liner 32 and the retaining strip 35. The retaining strip 35 is preferably the same material as the liner 32, but may alternatively be a high- or low-density polymer or another waterproof material that can be attached to the liner 32. The retaining strip 35 may further comprise self-luminescent material, such as Litroenergy™ particles.

The retaining strip 35 may be manufactured in a number of ways. The retaining strip 35 may be adhered to the liner 32, forming a envelope 36 at the bottom of the trough 23, between the liner 32 and the retaining strip 35. The retaining strip 35 may be adhered to the liner 32 by heat seal during the manufacturing process, or by application of an adhesive after the manufacturing process. The retaining strip 35 may alternatively be extruded integrally with the liner 32, such as when the retaining strip 35 and liner 32 are made of the same material or co-extrudable materials.

When the trough lining assembly is in place in the trough 23, the retaining strip 35 may be substantially parallel to the top of the trough 23, or may be concave with respect to the top of the trough 23, as shown in FIGS. 1a-b. The aerator 17 is retained in the envelope 36, at or near the bottom of the trough 23, so that it does not float to the top of the trough 23. To facilitate release of the aerating gas into the biomass, the retaining strip 35 may have air openings 37 cut into it, as shown in FIG. 3a. The air openings 37 may be slits or shapes, as shown in FIGS. 3a-d, and may be randomized or follow a pattern. The amount of aerating gas released into the biomass at certain points along the trough 23 may be controlled using a predetermined pattern of air openings 37. For example, fewer air openings 37 at the proximal end of the trough 23, where the biomass is deposited, will release less aerating gas, and air openings 37 are gradually added or enlarged, releasing an increasing volume of aerating gas into the biomass as it travels to the distal end of the trough 23.

In an alternate embodiment, the aerator 17 may be attached to the liner 32 by an adhesive. In another alternate embodiment, shown in FIGS. 2a-b, the trough lining assembly is fitted with a flat aerator 17 during the manufacturing process. The aerator 17 adheres to the liner 32 by heat seal due to the temperatures involved in the manufacturing process. The aerator 17 forms a triangle 16 with the bottom of the trough lining assembly into which the aerating gas is pumped under pressure.

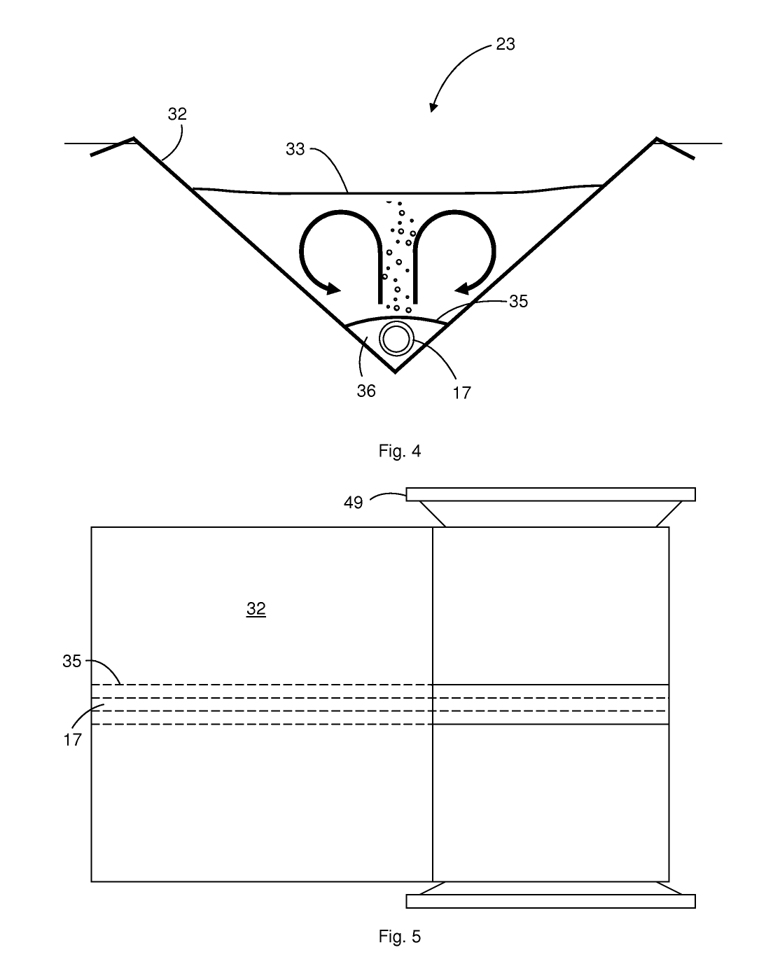

The aerator 17 preferably runs the entire length of the trough 23, so that the aerating gas is released substantially continuously along the length of the trough 23. The substantially continuous release induces a “churning” motive force in the biomass, shown in FIG. 4. The churning exposes more of the biomass to both sunlight and the aerating gas. The substantially continuous release also provides a consistent source of aerating gas that is absorbed or diffused within the biomass. For growth of algae, shrimp, or other organic material, the substantially continuous release of aerating gas provides the amount of aerating gas needed to maximize the growth benefits at all points in the trough. Further, for growth of organic material, as the biomass proceeds along the trough it will increase in concentration of organic material. The higher concentration will require more aerating gas. It is contemplated that the volume of gas released may continuously or periodically increase from the proximal end of the trough 23, where the biomass is deposited, to the distal end of the trough 23, where the biomass is harvested as explained below. In one embodiment, the aerating substrate 17 may have an increasing porosity from the proximal end to the distal end. In another embodiment, the aerating substrate 17 may be coated in a non-porous material that is gradually eliminated along the length of the aerating substrate 17.

The aerating gas may be injected before the trough is filled with biomass or after. The aerating gas may be atmospheric air, CO₂, or any combination of gases that facilitates the chemical reactions desired in the biomass. For the growth of algae or other plants, the aerating gas is preferably a mixture of CO₂-enriched air and NOₓ gas.

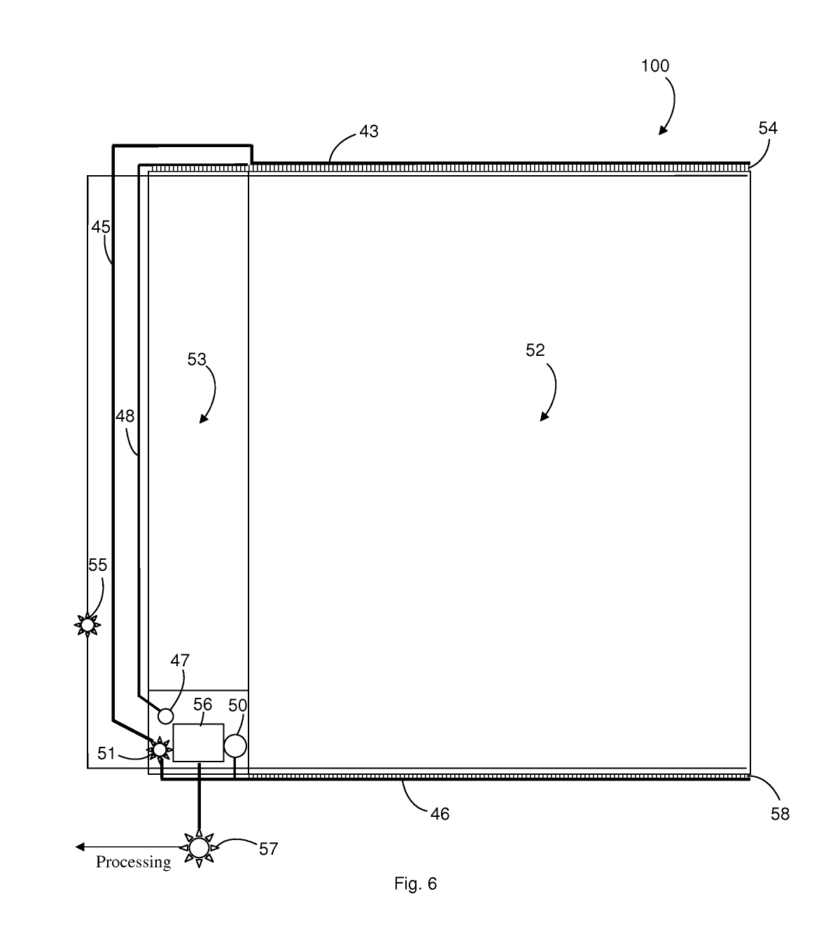

Referring to FIG. 5, the trough lining assembly is flat before deployment and can be rolled, fully assembled and without damage, onto a deploying spool 49. To install the trough lining assembly, a loaded deploying spool 49 may be mounted in a truck bed or other installation implement having a wheel base that straddles the trough 23. The trough lining assembly is then rolled off the deploying spool 49 and laid in the trough 23. In the preferred embodiment, one or more gas injectors is attached to the pressurizable, tubular aerator 17 at the proximal or distal end, or both ends, and an outlet line may be installed at the distal end of the trough, either onto or through the liner 32. The ends of the liner 32 are covered by dirt from the berms 20 once the trough lining assembly is in place.

The aerator 17 may have a shorter operating life than the liner 32. In the preferred embodiment, the aerator 17 may be replaced by simply attaching a new aerator to one end of the old aerator 17 and pulling the old aerator 17 out of the envelope 36 from the opposite end, simultaneously pulling the new aerator into place. The old aerator 17 may then be detached and discarded.

Once the trough lining assembly is laid in the trough 23, a solar cover 33 may be laid over the top as shown in FIGS. 1b-2b. The solar cover 33 is transparent or substantially translucent to allow sufficient sunlight to enter the biomass. Preferably, the solar cover 33 is made of 1-2 mil thick extruded polyethylene, which is substantially elastic and capable of floating freely on the surface of the biomass. The solar cover 33 may alternatively be held in place over the trough 23 by covering the ends of the solar cover 33 with dirt from the berms 20. The solar cover 33 may cover one or more troughs 23. In the preferred embodiment, the solar cover 33 covers a single trough 23. See FIG. 1b. In an alternate embodiment, the solar cover may cover a plurality of troughs 23.

The solar cover 33 initially lays flat over the troughs 23. As gas 40 collects within the trough lining assembly, the solar cover 33 is expandable to contain the volume of gas 40. The volume 40 does not interfere with the progression of the biomass through the system. If the ends of the solar cover are secured, such as by insertion into the berms 20 as shown in FIGS. 2a-b, the volume of gas 40 may be easily collected with a gas collection system.

During winter months, a second solar cover can be installed over the first solar cover. The second solar cover creates an environment where temperature can be maintained. The parasitic temperature loss of the biomass during winter months can be managed by the greenhouse effect where the biomass temperature would serve to heat the air, along with sunlight, between the upper and lower solar covers. One or both solar covers can be replaced seasonally to relieve excess heat during the summer months. The edges of the solar cover are covered with dirt using mulch-laying equipment. Tractors 28 can straddle each bioreactor bed to travel up and down the rows for periodic maintenance, repair of leaks, and replacement of the first or second solar cover. Alternatively, over-the-row tunnels or miniature greenhouses can be used for temperature control and durability during changing weather conditions.

Maintenance

The surfaces of a trough assembly that come in contact with then biomass may gradually accumulate film, which decreases efficiency of the system by obscuring sunlight and restricting flow. The system design anticipates this potential loss in efficiency by using a long, wide trough 23. The trough 23 dimensions ensure a sufficient surface area to prevent accumulation of film from affecting biomass flow or exposure to sunlight. The present trough assemblies may be implemented in lengths up to the preferred length of 1250 feet while maintaining system performance in all operating conditions over the operating life of the trough assembly. If it becomes necessary to remove accumulated film from the surfaces of the liner 32 and solar cover 33, the solar cover 33 may be retrieved by tractor or other implement, after which the liner 32 is scrubbed with a tractor-powered scrubbing implement, and fresh mulch 33 is laid. Alternatively, the liner 32 may be scrubbed by depositing floating, textured balls, such as brushy balls, into each trough 23 at the proximal end. The balls loosen accumulated film on the liner 32 before they are retrieved at the distal end of the trough 23.

Algae Production Facility

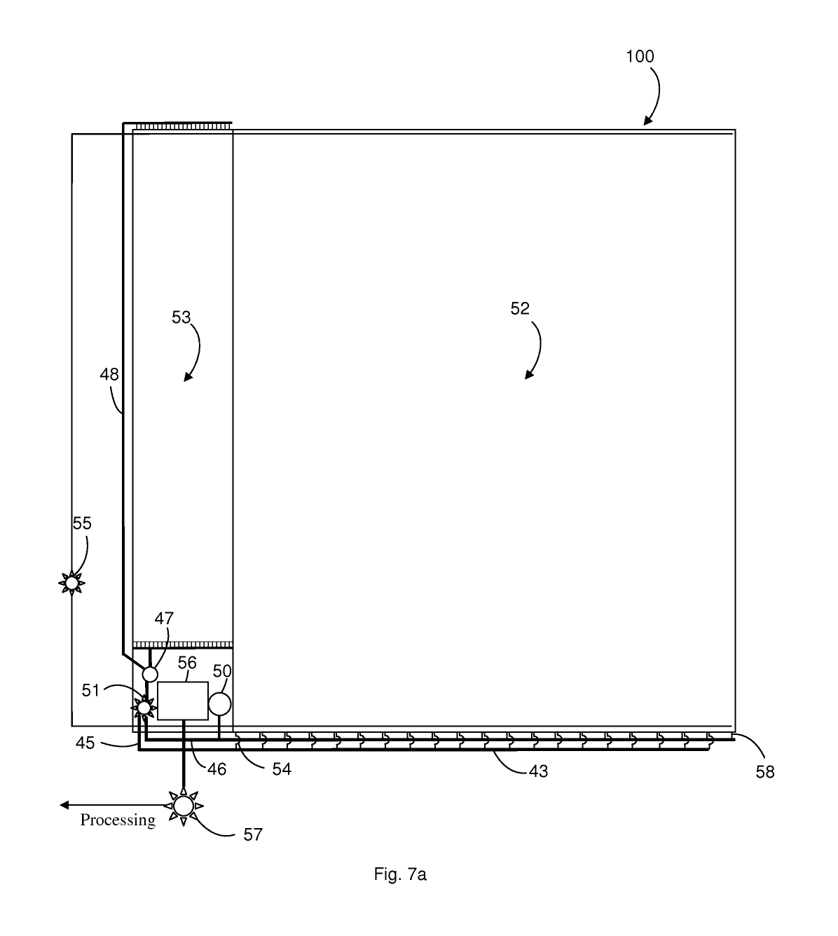

Referring to FIG. 6, an algae production facility includes at least one field 100 of beds comprising parallel troughs 23 separated by berms 20. The number and size of fields 100 are limited by the land available, cost and other factors. For large scale algae production, a series of fields 100 will be interconnected into a common algae collection point for ease of processing. A field 100 is supplied by a harvest sump 50, circulation pump 51, inoculation sump 47, settling tank 56, and aerating gas injection system 55. Each field 100 is designed to provide an adequate dwell time for the algae to convert the injected aerating gas into O₂ through the photosynthesis process by exposing the algae to sunlight.

The troughs 23 are subjected to a “dead-leveling” procedure which ensures that the troughs 23 are uniform in dimension and parallel or identically graded with respect to the ground, so that a consistent biomass level may be maintained across all trough lining assemblies. Once the troughs 23 are substantially uniform and parallel, a tractor 28, pickup truck, or other installation implement lays the preferred trough lining assemblies into the troughs 23. The tractor 28 also lays the solar cover 33 over the troughs 23 if the temperature maintenance, weather protection, or gas collection benefits of the solar cover 33 are desired.

The trough lining assemblies are connected to a common inlet line 45 and outlet line 46, a circulation pump 51, control valves (not shown), and one or more aerating gas injection pumps 55. Biomass is introduced to the facility at the circulation pump 51, which pumps the biomass through the system. From the circulation pump 51, the biomass travels through the inlet line 45, into the inlet header line 43, which connects to each trough. The biomass is deposited into the trough lining assemblies in the growout troughs 52 through an inlet valve 54 in each trough. Aerating gas is injected under pressure into the aerator 17, which pressurizes into its tubular shape. Once pressurized, the aerator 17 gradually releases aerating gas into the biomass stream through the air openings 37 in the retaining strip 35. The aerating gas also agitates the biomass, keeping the aerating gas in suspension for a higher conversion rate of CO₂ to O₂ and churning the biomass to increase algal exposure to sunlight. As the biomass travels the length of the trough assembly, the algae concentration increases, as does CO₂ intake and O₂ output. The increasing volume of O₂ and water vapor may expand the solar cover 33, if present, and the O₂ may be collected through a gas collection valve at or near the end of the trough assembly. At the distal end of the trough, the biomass passes through an outlet valve 58 into the output line 46 and is either diverted to the harvest sump 50 or continues to the circulation pump 51 for recirculation, as described below.

As shown in FIG. 6, the preferred embodiment of a field 100 of 40 gross acres (1320 ft×1320 ft) has 121 1250 ft-long growout troughs 52; 15 inoculation troughs 53; 36 net acres of trough beds (1250 ft×1250 ft); over 19 net acres of biomass surface area (1250 ft×60 in.×135); a capacity of approximately 4.8 million gallons; a flow rate of about 3300 gpm/field or about 24 gpm/trough; and algae dwell time of 24 hours. At this dwell time, the biomass travels through the trough at a velocity of 0.808 feet per minute. The inoculation troughs 53 are fed by an inoculation line 48 connected to the inoculation sump 47. The desired dominant species of algae is grown in the inoculation troughs, which are operated independently of the growout troughs 52. Inoculated biomass is circulated through the inoculation sump 47 and diverted to the circulation pump 51 as needed to maintain dominance of the preferred species of algae in the growout troughs 52.

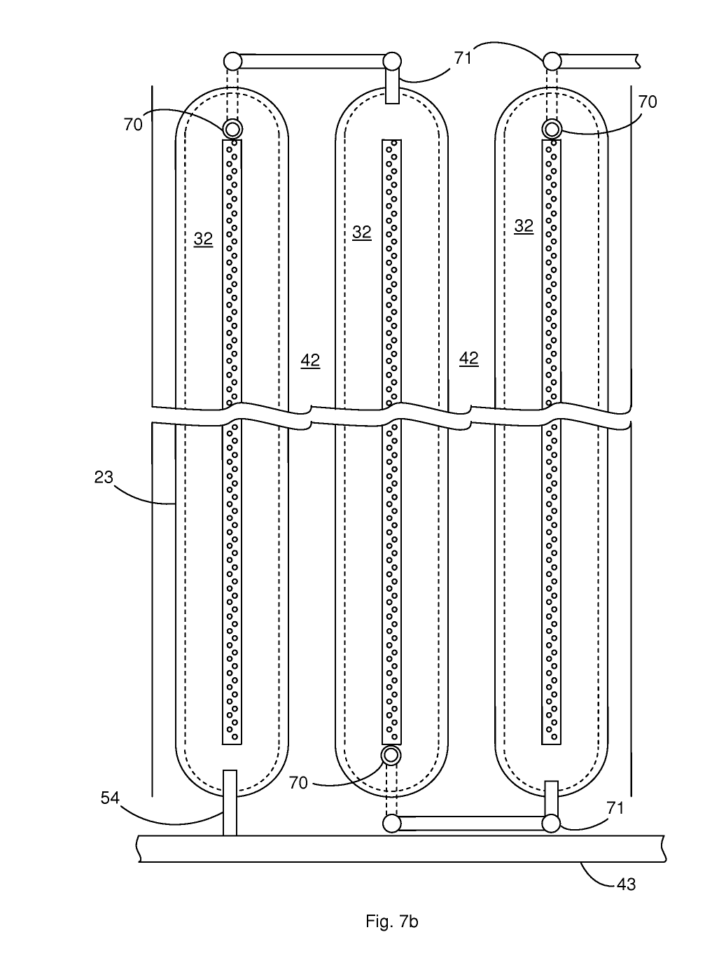

In some environments, a higher flow velocity may be desirable to add motive force to the algae, preventing it from accumulating on the trough lining assembly 31. The alternate embodiment of a field 100 shown in FIG. 7a has the same trough 23 dimensions as the preferred embodiment, but provides an increased flow velocity in the growout troughs 52 by connecting adjacent troughs and allowing the biomass to flow through multiple troughs before passing into the output line 46. The connection between adjacent troughs 23 allows the biomass to flow in alternating directions. In the example shown in FIG. 7b, the biomass enters a drain 70 that passes through the liner 32 and into the ground at the distal end of one trough 23. The biomass travels through a siphon 71 and is deposited at the proximal end of the next trough 23. The connection between troughs 23 may also be facilitated by mechanical pumps. After a predetermined number of troughs 23, the biomass is let into the output line 46 through an outlet valve 58. Any number of troughs may be connected to each other between an inlet valve 54 and an outlet valve 58. Preferably, the biomass travels the length of six troughs before release, which results in a flow velocity of 5.2 feet per minute at a dwell time of 24 hours.

Algae Production Cycle

In the algae production cycle, the facility is initialized with biomass and growth is encouraged by maintaining proper algae, CO₂, and fertilizer concentrations, as well as sunlight and temperature. The harvest process begins when the biomass reaches sufficient concentration, referred to herein as “harvest concentration.” To harvest algae from the field 100, a partial diversion of the biomass is initiated. Between 20% and 80% of the biomass, depending on the present concentration, may be diverted daily for harvesting algae. The diverted biomass is delivered to a harvest sump 50 while the remaining biomass, called the bypass biomass, continues through the facility to the circulation sump 51. In the harvest sump 50 a flocculant may be added to the diverted biomass to facilitate settling of the algae. The flocculant may be any known agent that will encourage flocculation without killing or harming the algae. Preferably, the flocculant is a commercially produced polyacrylamide or a natural product such as chitosan.

The diverted biomass is then delivered to a settling tank 56. The settling tank 56 is preferably a weir tank, which will facilitate settling of the algae. Once the algae settles, it is collected by a harvest pump 57. The water remaining in the settling tank 56 is delivered to the circulation pump 51, where it is mixed with the bypass biomass to dilute the biomass that is reentering the field 100. This recirculated water contains byproducts of the previous algae growth process, such as salt and fertilizer, that are beneficial to subsequent growth processes. The biomass will therefore be comprised of recirculated water in amounts necessary to optimize algae production and maintain the biomass at an ideal range of concentration. The solids content percentage in the biomass is measured periodically to make sure it is not exceeding a pre-determined limit. Excess concentration is easily controlled with the introduction of chlorine or simple dilution. While the harvest cycle is continuous, the total volume will vary throughout the seasons of the year.

The harvest pump 57 may have a filter to create an algae cake for easy harvest and transportation. After the algae is harvested, it is further processed for its desired use. For example, the wet algae may be subjected to processing methods which efficiently extract algae oil. The efficiency is created when the algae can be processed on-site without the need to dry and transport the material. However, in another example, the algae it may be dried, on-site, into a product which facilitates storage and shipping, so that the dry algae may be sold to customers who will process it according to their needs.

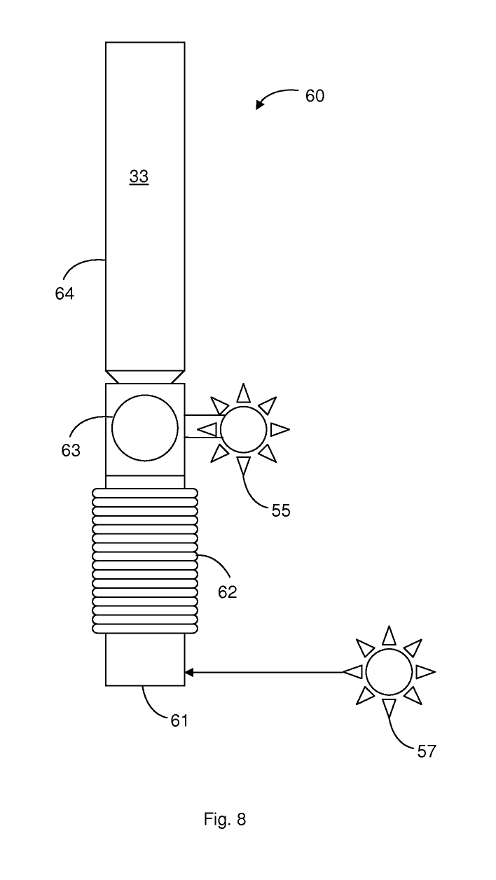

In one embodiment of an on-site dryer 60, shown in FIG. 8, the harvested algae is deposited onto a conveyor 61 that slowly transports the algae through a drying tunnel 62. Hot air is injected at a high velocity opposite the direction of the conveyor 61, so that the algae is dry by the time it has traveled the length of the drying tunnel 62. The hot air for drying is supplied by a propane furnace 63. To increase the efficiency of the facility, CO₂ and NOₓ gases generated by combustion within the propane furnace 63 are vented over a heat exchanger into the aerating gas injection pump 55, enriching the atmospheric air to be injected into the aerator 17. Since a standard propane furnace 63 can only increase the temperature of atmospheric air a limited amount, the efficiency of the dryer 60 can be further increased by supplying preheated air to the propane furnace 63. The preheated air is obtained from an air trough 64 and covered by a solar cover 33, creating a greenhouse effect that heats the air before it is delivered to the propane furnace 63. The air trough 64 may have the same dimensions as a trough 23 so that it may be created and maintained with the same implements used to create and maintain the troughs 23. The air trough 64 and dryer 60 may be in-line with the troughs 23 in a field 100 to maintain continuity of the field design.

The gas 40 produced by the algae is primarily O₂. The gas 40 may be collected and processed depending on the overall configuration of the system. In one embodiment, the facility is placed in proximity and connected to a factory that burns oxygen during production and expels CO₂ and other gases. The factory provides the system with CO₂, which is pressurized and injected into the trough assemblies. The collected gases 40 then represent the amount of CO₂ emission from the factory that has been scrubbed of carbon. This amount can be tested and the data used by the factory to show reduction of polluting emissions. After testing, the O₂ may supply the factory's burners to increase production efficiency. In another embodiment, livestock manure and food waste can be recycled to produce CO₂ for injection into the system.

Production is affected primarily by the number of daylight hours. To overcome seasonality of the production system and provide a constant supply of biomass for processing 24 hour 7 day per week, the number of fields 100 required is determined by the output on the day with shortest daylight hours of the year. As the volume increases with longer daylight hours, unnecessary fields can be idled.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

We claim:

1. A method of aerating a biomass, the method comprising:
a. laying a trough lining assembly in a trough having a top, a bottom, a distal end, and a proximal end, the trough lining assembly comprising:
i. a liner and;
ii. an aerator;
b. depositing the biomass into the trough; and
c. injecting an aerating gas into the trough at the proximal end near the bottom of the trough, such that the aerating gas is released through the aerator substantially continuously along the length of the trough.

2. The method of claim 1 further comprising replacing the aerator with a new aerator without removing the biomass or liner from the trough.

3. The method of claim 2 wherein the trough lining assembly further comprises a retaining strip attached to the liner such that the retaining strip and liner form an envelope in which the aerator is retained near the bottom of the trough.
